(19) United States
(12) Patent Application Publication
(10) Pub. No.: US 2015/0359520 A1
(43) Pub. Date: Dec. 17, 2015

(54) ULTRASOUND PROBE AND ULTRASOUND IMAGING SYSTEM

(71) Applicant: KONINKLIJKE PHILIPS N.V., Eindhoven (NL)

(72) Inventors: CAIFENG SHAN, EINDHOVEN (NL); JOZEF HUBERTUS GELISSEN, EINDHOVEN (NL); SEBASTIAN MUÄELER, EINDHOVEN (NL)

(21) Appl. No.: 14/762,364

(22) PCT Filed: Jan. 14, 2014
(86) PCT No.: PCT/IB2014/058248

§ 371 Date: Jul. 21, 2015

Related U.S. Application Data
(60) Provisional application No. 61/755,090, filed on Jan. 22, 2013.

(51) Int. Cl. A61B 8/08; A61B 8/00

(52) U.S. Cl. CPC A61B 8/5223; A61B 8/4461; A61B 8/4472; A61B 8/0858; A61B 8/4254

(57) ABSTRACT

The present invention relates to an ultrasound probe (10) for an ultrasound imaging system (100), comprising: —a probe housing (40), —a single element ultrasound transducer (26) for transmitting and receiving ultrasound signals, —a transducer movement unit (48) arranged within the probe housing (40) for moving the single element ultrasound transducer (26) relative to said probe housing (40) along a two-dimensional convex curved pathway during signal acquisition.

### FIELD OF THE INVENTION

The present invention relates to an ultrasound probe for an ultrasound imaging system. The present invention further relates to an ultrasound imaging system that comprises such an ultrasound probe. Even further the present invention relates to a method of determining an amount of visceral body fat of a subject (patient) and a corresponding computer program for implementing the method.

### BACKGROUND OF THE INVENTION

In the field of performance sports, personal fitness and health care appliances it is desirable to get insight into a body's proportional composition of different tissue types. For this purpose it is necessary to distinguish several main tissues from each other. The most important tissues to detect from a health perspective are: fat mass and fat-free mass, lean body mass and muscle mass and a further discrimination of subcutaneous adipose tissue (SAT) and visceral adipose tissue (VAT).

Fat storage can roughly take place in two different compartments of the human body: subcutaneous (underneath the skin) and visceral/intra-abdominal (surrounding the internal organs). VAT is harder to loose than SAT and is considered to be more dangerous. Studies have shown that people with high visceral fat are more susceptible to heart disease, stroke, diabetes and hypertension. Sedentary people, smokers and drinkers have been shown to have more VAT than active people who are non-smokers and non-drinkers. Stress may also be a factor in the storage of VAT in the body.

Medical professionals have to increasingly deal with the above-mentioned diseases that relate from high amounts of VAT. Having a method for quickly and reliably assessing a patient's level of physical fitness can help the professional to assess to what extent the physical fitness may be impacting the patient's health. Moreover, medically prescribed exercise intervention with fitness level and disease monitoring could be used to improve the patient's health and also document the effectiveness of the treatment. A direct quantification of VAT is however difficult.

Most of today's known methods for quantifying VAT rely on estimation rather than on direct and accurate quantification methods. An easy method to obtain the VAT of a person is the measurement of the waist circumference. However, this parameter has several limitations, since it includes the less harmful subcutaneous fat (SAT) and the muscle layers of a person as well.

Another way of measuring a person's body fat percentage is by measuring the weight and volume of the person to find average tissue density. Together with assumptions on bone mass and knowing the densities of muscle and fat, a body fat measurement can be calculated. This method is fairly consistent over multiple measurements. Unfortunately, the procedure involves submerging the subject in a water tank, making the method bulky and time-consuming. Apart from that, it does also not allow to accurately distinguish between SAT and VAT.

Therefore, a direct quantification of human VAT is required. Several scientists proposed to make use of the so-called intra-abdominal diameter (IAD) as a valid means to estimate the human VAT. The IAD describes the distance between the Linea Alba, 3 cm above the umbilicus on the L3-L4 level of the spine, and the posterior side of the Aorta. Bellesari et al.: “Sonographic measurement of adipose tissue”, Journal of Diagnostic Medical Sonography, January 1993, Vol. 9, No. 1, 11-18, confirmed the potential of the IAD, however also strengthened the fact that the IAD is not very reliable, most likely due to the differences in pressure applied with the ultrasound probe. Additionally, they reported repeatability issues due to pulsating effects of the Aorta, as well as respiratory and intestinal motion. Furthermore, they reported some scans to have issues with shadows (dark areas) or reflections (regularly spaced, thin, bright lines) interfering with the recognition of IAD. These problems were usually corrected by applying more ultrasound gel. A concept to overcome these issues would be a desirable step towards a better and more direct access to information about a person's VAT. Tornaghi et al.: “Anthropometric or ultrasonic measurements in assessment of visceral fat? A comparative study”, International Journal of Obesity, 1994 (18), 771-775 compared the accuracy of anthropometric and ultrasonic measurements in assessing the amount of visceral adipose tissue.

A consumer product that allows to quantify a person's VAT in an easy way would be especially desirable. Since state of the art medical ultrasound imaging technology is too expensive for a consumer product, low-cost solutions are required. Medical ultrasound imaging systems that are designed for the professional sector are apart from that too difficult to handle for a private consumer in daily use.

### SUMMARY OF THE INVENTION

It is an object of the present invention to provide an ultrasound probe for an ultrasound imaging system that is particularly suitable for private consumers, easy in handling and, compared to state of the art products, less cost-intensive. Preferably, such device shall be configured to be easily and conveniently operated in a home setting. It shall allow a direct and for the consumer easy to handle quantification of human VAT. It is furthermore an object of the present invention to provide a corresponding ultrasound imaging system as well as a corresponding method for determining an amount of visceral body fat of a subject from an abdominal ultrasound scan.

In a first aspect of the present invention an ultrasound probe for an ultrasound imaging system is presented. The ultrasound probe comprises:

a probe housing,

a single element ultrasound transducer for transmitting and receiving ultrasound signals,

a transducer movement unit arranged within the probe housing for moving the single element ultrasound transducer relative to said probe housing along a two-dimensional convex curved pathway during signal acquisition.

In a further aspect of the present invention an ultrasound imaging system is provided that comprises the above-mentioned ultrasound probe and an image reconstruction unit for reconstructing an ultrasound image from the received ultrasound signals.

In a still further aspect of the present invention a method of determining an amount of visceral body fat of a subject from an abdominal ultrasound scan is provided. The method comprises the steps of:

receiving ultrasound signals from a single element ultrasound transducer that is during signal acquisition automatically moved within a probe housing of an ultrasound probe along a convex curved pathway,

reconstructing an ultrasound image from the received ultrasound signals,

segmenting the ultrasound image of the abdominal ultrasound scan of the subject,

identifying a position of a Linea Alba and an Aorta within said ultrasound image in order to derive an intra-abdominal diameter (IAD), and

calculating the amount of visceral body fat based on the derived IAD.

In a still further aspect of the present invention a computer program is presented comprising program code means for causing a computer to carry out the steps of such method when said computer program is carried out on the computer.

As it has been mentioned above, it is possible to calculate/estimate the amount of human VAT based on the intra-abdominal diameter (IAD). This requires the identification of the Linea Alba and the Aorta within the ultrasound image. Since it is difficult to detect the Linea Alba or the Aorta from a one-dimensional ultrasound signal (A-mode), a two-dimensional ultrasound image is needed. Two-dimensional ultrasound images are usually acquired directly by a multiple-element ultrasound transducer array. Ultrasound probes equipped with such multiple-element ultrasound transducer arrays are, however, quite costly.

The present invention is based on the idea to provide a single element ultrasound transducer, wherein the single element ultrasound transducer is moved automatically relative to the probe housing along a two-dimensional convex curved pathway during signal acquisition. This is accomplished by a transducer movement unit that is arranged within the probe housing. During movement of the single element ultrasound transducer along the convex curved pathway, multiple ultrasonic A-line signals are collected. These one-dimensional ultrasound signals are then reconstructed to a two- or three-dimensional ultrasound image. The moved single element ultrasound transducer therefore covers a similar area as a multiple-element ultrasound array probe. The moved single element ultrasound transducer in other words imitates the shape of a convex array transducer.

One of the major advantages of the presented ultrasound probe is that only one ultrasound element is needed. Such an ultrasound probe is of course less expensive than a multiple-element array. It, however, allows to generate a two-dimensional ultrasound image that is comparable to images produced with convex (multiple-element) transducer arrays. During signal acquisition the single element transducer moves laterally along the described arc-shaped (convex) pathway, wherein said arc preferably has an opening angle between 0°-90°, most preferably between 45°-75°. The accessible range may thus be quite large, i.e. the scan range of a scanning sequence (transducer element moving forth and back) without moving the ultrasound probe relative to the examination object is quite large. Handling of the ultrasound probe is relatively easy, which makes it accessible for personal use (less-experienced private users). Since the single element ultrasound transducer is moved relative to the probe housing automatically (e.g. using an electromotor) and delivers “regular” 2D ultrasound images, a user might not even recognize the difference between the presented ultrasound probe and a “regular” multiple-element ultrasound array probe.

The ultrasound probe preferably also comprises a movement sensor for sensing a movement and/or position of the single element ultrasound transducer relative to the probe housing. This position tracking is especially important in order to be able to reconstruct the 2D B-mode image from the plurality of 1D scans taken during the movement of the transducer element along the above-mentioned arc. Preferably, transmit pulses are only sent when the movement sensor detects that the transducer element is in motion.

Preferred embodiments of the invention are defined in the dependent claims. It shall be understood that the claimed ultrasound imaging system and the claimed method have similar and/or identical preferred embodiments as the claimed ultrasound probe and as defined in the dependent claims.

In a preferred embodiment the transducer movement unit comprises a convex shaped rail for mechanically guiding the single element ultrasound transducer relative to the probe housing along the convex curved pathway. Said guiding rail may be part of a frame that is arranged and fixed within the probe housing. Preferably at least two such guiding rails are used, one on each side of the single element ultrasound transducer. The single element ultrasound transducer is preferably slidably mounted within said guiding rail. Different kinds of driving mechanisms are generally conceivable to move the single element transducer within the guiding rail, e.g. an electromotor, a magnetic drive train, etc.

In a further preferred embodiment the ultrasound probe further comprises a displacement sensor for sensing a movement and/or position of the single element ultrasound transducer and/or the probe housing. This displacement sensor is preferably realized as an optical sensor. The optical sensor may, for example, be attached to the ultrasound probe for optically detecting movements of said probe. This provides a simple and cost-effective, but precise way of obtaining movement or position information of the ultrasound probe. If coupled with the above-mentioned movement sensor for sensing the movement and/or position of the single element ultrasound transducer relative to the probe housing, it allows to determine the absolute position of the single element ultrasound transducer at each point in time in a very precise way.

In a further embodiment the ultrasound probe comprises at least one pressure sensor for sensing a pressure with which the ultrasound probe is pressed against a surface of an examination object. This pressure sensor may, for example, be arranged on or within a contact surface of the probe housing with which the examination object is contacted during signal acquisition. Such a pressure sensor especially has the advantage that differences in the ultrasound image resulting from different applied pressures may be accounted for. The pressure sensor may also be coupled with a visual, audible and/or tactile feedback unit for providing a feedback to the user about the pressure measured with the pressure sensor. In this case the user may receive an indication if the applied pressure is too high or too low. An audible warning signal may, for example, be generated if the user presses the ultrasound probe (i.e. the probe housing) against the examination object with a too high pressure that could negatively interfere the fat measurements. Alternatively, a green light may be provided on the probe housing that turns into a red light if the applied pressure is too high. Such an embodiment is especially advantageous to assist inexperienced users.

In a further embodiment of the present invention the probe housing has a three-dimensional convex curved contact surface for contacting a surface of an examination object, wherein the contact surface is symmetric with respect to an imaginary vertex line that divides the contact surface in two identical halves. The contact surface is, of course, not effectively divided in two halves but forms a continuous contact surface. The described imaginary vertex line is herein only included for illustrative purposes. The vertex line is arranged on top of the arc-shaped (convex curved) contact surface in its middle.

According to a preferred embodiment the ultrasound probe comprises two pressure sensors for sensing a pressure with which the probe housing is pressed against a surface of an examination object, said two pressure sensors being arranged on the imaginary vertex line and spaced apart from each other.

As already mentioned above, the applied pressure at the interface between the probe housing and the examination object is an important factor that needs to be sensed/controlled in order to achieve reproducible results. Having two pressure/force sensors that are spaced apart from each other and arranged on the imaginary vertex line of the convex curved contact surface has an important advantage, since it allows to measure whether the ultrasound probe (probe housing) is placed perpendicular in cranial/caudal (up/down). If the probe housing is arranged perpendicular to the top surface of the examination object, the pressures measured by the two pressure sensors should be equal. To support the user in handling the device correctly, the above-mentioned feedback unit may provide a visual, audible and/or tactile feedback to the user whether the pressures of both sensors are the same (i.e. the probe head of the probe housing is arranged correctly (perpendicularly)) or not.

The longer the distance between the above-mentioned two pressure sensors, the more robust is the measurement. In other words, if the distance between the two pressure sensors is quite large, it can be accurately detected if the ultrasound probe is arranged perpendicular to the top surface of the examination object. According to an embodiment of the present invention it is therefore preferred that both pressure sensors are arranged on or within the contact surface on two opposing sides of the contact surface (and on the above-mentioned imaginary vertex line), wherein a distance between the two pressure sensors substantially equals a width of the contact surface.

In an alternative embodiment the presented ultrasound probe comprises three pressure sensors that are arranged on or within the contact surface for sensing a pressure with which the ultrasound probe is pressed against a surface of an examination object, wherein a first pressure sensor is arranged on the imaginary vertex line, and wherein a second and a third pressure sensor are spaced apart from the first pressure sensor and equally spaced apart from said vertex line.

In contrast to the above-mentioned first alternative, three pressure sensors are provided instead of two. One of the three pressure sensors is therein still arranged on the vertex line, i.e. in the middle of the contact surface of the ultrasound probe housing, while the other two sensors are equally spaced apart from said vertex line to the left and right. This embodiment has the advantage that it allows to not only sense whether the transducer housing is arranged perpendicular to the top surface of the examination object in cranial/caudal direction, but also to sense whether it is arranged perpendicular in lateral (left/right) direction. To check whether the transducer is perpendicular in cranial/caudal direction, the pressure of the first pressure sensor (that is arranged on the imaginary vertex line) has to equal the sum of the second and third pressure sensors. To check whether the probe housing is arranged perpendicular in lateral direction, the pressure of the second sensor has to equal the pressure of the third sensor.

A distance between the second and the third sensor may be in a range of a few millimeters, preferably in a range of 2-10 mm. The above-mentioned feedback unit may also in this embodiment produce a feedback that supports the user to correctly arrange the probe housing relative to the examination object (perpendicular in both directions).

In a still further embodiment the ultrasound probe additionally comprises two capacitive sensors arranged on two opposing lateral sides of the contact surface for sensing if the probe housing makes contact with an examination object over the whole contact surface, wherein a distance between the two capacitive sensors substantially equals a length of said contact surface. The two capacitive sensors are preferably arranged on the lateral sides of the contact surface and not on the upper and lower sides of the contact surface, where the above-mentioned pressure sensors are arranged. An imaginary line that connects the two capacitive sensors may, for example, be perpendicular to the imaginary vertex line. In other words, the contact surface preferably has a rectangular shape when seen in a top view, wherein the two capacitive sensors are arranged on the two short sides of said rectangle and the two or three pressure sensors are arranged on the two long sides of the rectangle.

As already mentioned above, the present invention does not only refer to the ultrasound probe itself, but also to an ultrasound imaging system comprising such an ultrasound probe and an image reconstruction unit for reconstructing a 2D or 3D ultrasound image from the received ultrasound signals.

In a preferred embodiment said ultrasound imaging system further comprises:

an identification unit for identifying a reference point within the reconstructed ultrasound image, and

a focussing unit for focussing the single element ultrasound transducer on the reference point during a movement of the ultrasound probe relative to an examination object.

The main purpose of this ultrasound imaging system is the measurement of visceral adipose tissue (VAT). The ultrasound imaging system is in practice preferably applied as follows: The user places the ultrasound probe just above the umbilicus at the L3-L4 level of the spine. Preferably, a predefined pressure is applied to the belly of the patient with the ultrasound probe, wherein said predefined pressure is measured via the one or more pressure sensors mentioned above. In the next step, the single transducer element will start moving (sweeping) laterally along the convex curved pathway in order to image the epigastric/umbilical region at L3-L4 level of the spine. During said first signal acquisition, the ultrasound probe shall be hold still (not moved), wherein only the single element transducer is moved relative to the probe housing. The image reconstruction unit reconstructs a two-dimensional ultrasound image from the received ultrasound signals. Due to the convex curved pathway, this image will have a cone shape, similar to ultrasound images taken with a multi-element arc-shaped transducer heads.

The identification unit will then identify a reference point within the reconstructed ultrasound image by applying an image analysis algorithm. A preferred reference point is the Aorta. The Aorta is easy to identify within the reconstructed ultrasound image, as it usually represents the largest pulsating object within the image. An image analysis algorithm may thus identify the Aorta relatively easily. As soon as the Aorta has been identified, the user may receive a feedback that the ultrasound probe may now be moved over the belly in a horizontal plane in order to receive further image sequences. During this manual movement of the ultrasound probe the focusing unit will automatically keep the focus on the Aorta as reference point. The above-mentioned displacement sensor senses the movement and/or position of the single element ultrasound transducer and/or the probe housing during this time. Images may be either taken in real time during the probe movement or at distinctive points where the user arranges the ultrasound probe relative to the patient on different points of the belly. In this way several image sequences may be taken to image the whole epigastric/umbilical region. The image reconstructing unit may reconstruct a full body scan of the whole region by combining the plurality of scans with each other to finally visualize an ultrasound image of the complete epigastric region.

The ultrasound imaging system may, according to a further preferred embodiment, additionally comprise a segmentation unit for segmenting the ultrasound image of the abdominal ultrasound scan of the subject and for identifying the position of the Linea Alba and the Aorta within said ultrasound image in order to derive the intra-abdominal diameter (IAD), and a calculation unit for calculating an amount of visceral body fat based on the derived IAD.

The image analysis unit may apply an image analysis algorithm that is adopted to derive the location of the Linea Alba and the Aorta. In a first step this usually comprises a region of interest (ROI) selection. An ROI for the Linea Alba detection and an ROI for the Aorta detection can be first selected in the original input ultrasound image. The ROIs can be selected based on prior knowledge of the anatomical structure and ultrasound imaging. For example, the Linea Alba will lie in the upper part of the ultrasound image and the Aorta is represented by the largest pulsating object in the middle part of the image.

To increase the contrast of the gained ultrasound image, image enhancement techniques are applied to the selected ROIs in a next step. For example, a histogram equalization can be adopted to enhance the contrast by spreading out the most frequent pixel intensity values. Such an image enhancement technique is, for example, known from S. H. Contreras Ortiz, et. al.: “Ultrasound image enhancement: A review”, Biomedical Signal Processing and Control, 7(5): 419-428, 2012.

Object localization techniques are then adopted to localize the Linea Alba and the Aorta in the processed ROIs. Different methods exist for object localization in computer vision and image analysis areas. In one embodiment, machine learning based methods can be used. Given many positive samples (e.g., the image patch of Linea Alba) and negative samples (e.g., the image patch not corresponding to Linea Alba), machine learning techniques are used to train a detector for Linea Alba or Aorta. Such a machine learning technique is exemplarily described in P. Viola and M. Jones: “Rapid Object Detection using a Boosted Cascade of Simple Features”, CVPR conference 2011. With the trained detector, a given ROI is scanned at multiple scales and multiple positions, to check whether the Linea Alba (or Aorta) exists in the ROI and if yes, to find the location.

In another embodiment, a deformable template model can be considered (see e.g. A. K. Jain, et al.: “Object Matching Using Deformable Templates”, IEEE Trans. Pattern Analysis and Machine Intelligence, 18(3): 267-278, 1996). A prototype template is defined for the Linear Alba or the Aorta based on the prior knowledge. Given a ROI, the template is applied to multiple positions (at multiple scales), to see whether the Linea Alba (or Aorta) can be matched. Based on template matching, the Linea Alba or Aorta may be localized.

In case multiple ultrasound images (or video) are acquired, information from multiple frames can be combined to improve the accuracy and robustness. In one embodiment, the detection results in multiple images are combined (e.g., averaging) to derive the final location of the Linea Alba or Aorta, which is a decision-level fusion. In another embodiment, feature-level fusion can be used, that is, image content (or features) of multiple images are considered in the object localization.

If the Linea Alba and the Aorta are finally detected within the ultrasound image, the IAD (linear distance between the Linea Alba and the posterior wall of the Aorta) can be derived. The calculation unit may then calculate the amount of visceral body fat based on the derived IAD.

### BRIEF DESCRIPTION OF THE DRAWINGS

These and other aspects of the invention will be apparent from and elucidated with reference to the embodiment(s) described hereinafter. In the following drawings

FIG. 1 shows a schematic illustration of an ultrasound imaging system according to an embodiment of the present invention;

FIG. 2 shows a schematic block diagram of the ultrasound imaging system according to an embodiment of the present invention;

FIG. 3 shows a perspective view (FIG. 3A) and a top view (FIG. 3B) of an ultrasound probe according to an embodiment of the present invention;

FIG. 4 shows several embodiments of the ultrasound probe in a front view;

FIG. 5 shows a schematic representation of a human abdominal region to illustrate the intra-abdominal diameter (IAD);

FIG. 6 schematically illustrates the scanning procedure with the ultrasound probe according to the present invention;

FIG. 7 shows a further block diagram illustrating several further components of the ultrasound imaging system according to a further embodiment of the present invention; and

FIG. 8 shows a schematic flow diagram of a method according to an embodiment of the present invention.

### DETAILED DESCRIPTION OF THE INVENTION

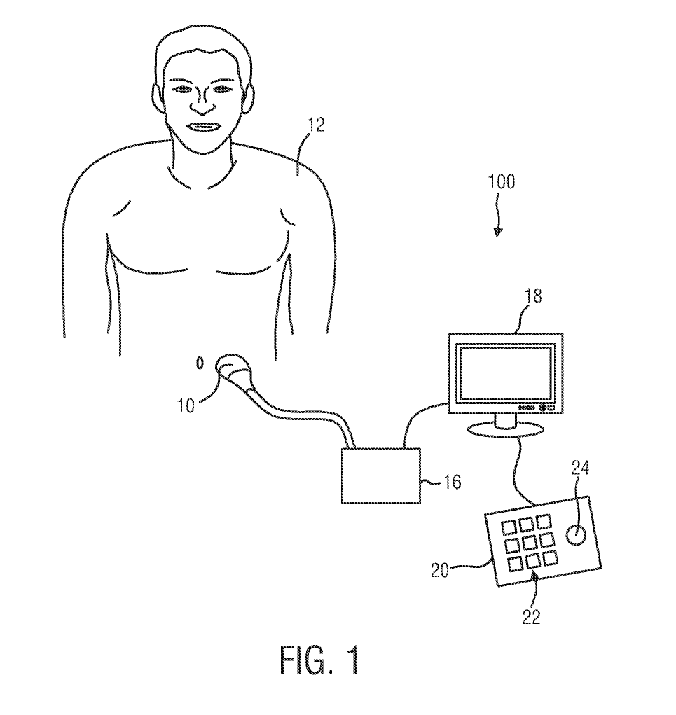

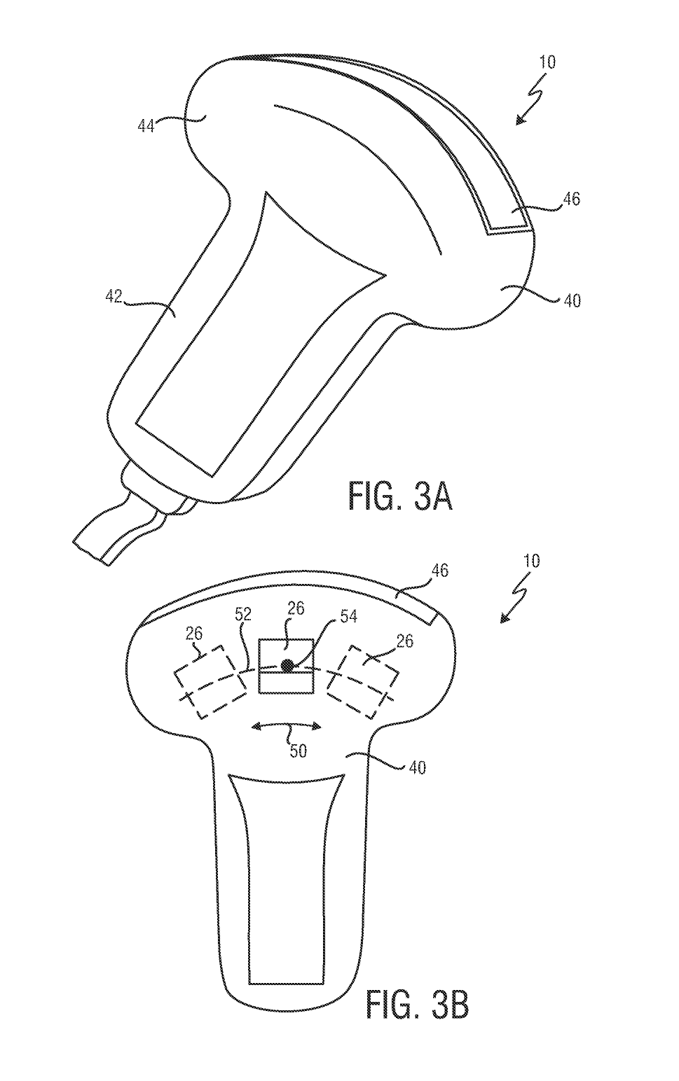

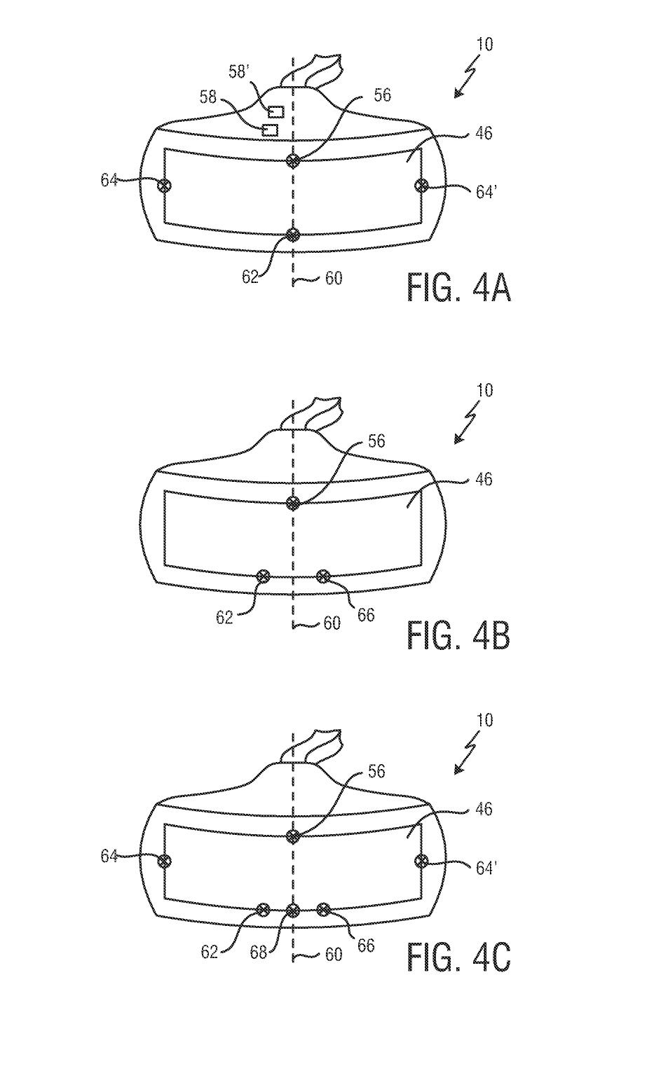

FIG. 1 shows a schematic illustration of an ultrasound imaging system 100 according to an embodiment of the present invention. The ultrasound imaging system 100 is applied to inspect a volume of an anatomical site, in particular an anatomical site of a subject 12 (e.g. a patient 12). The ultrasound imaging system 100 comprises an ultrasound probe 10 for transmitting and receiving ultrasound signals. The details of said ultrasound probe 10 will be explained in detail further below with reference to FIGS. 3 and 4. The ultrasound probe 10 may be hand-held by the user of the system, for example medical staff or a doctor. The presented ultrasound imaging system 100 is designed to be easy in use, such that also private persons may apply the system 100.

The ultrasound imaging system 100 further comprises a controlling unit 16 that controls the provision of an ultrasound image via the ultrasound imaging system 100. As will be explained in further detail below, the controlling unit 16 controls not only the acquisition of data via the ultrasound transducer of the ultrasound probe 10 but also signal and image processing that form the resulting ultrasound images out of the echoes of the ultrasound beams received by the ultrasound transducer that is integrated in the ultrasound probe 10.

The ultrasound imaging system 100 further comprises a display 18 for displaying the received ultrasound images to the user. Still further, an input device 20 may be provided that, for example, comprises keys or a keyboard 22 and further inputting devices, for example a trackball 24. The input device 20 may either be connected to the display 18 or directly to the controlling unit 16.

It shall be noted that FIG. 1 is only a schematic illustration. Appliances in practice may deviate from the concrete design shown in FIG. 1 without leaving the scope of the invention. The ultrasound probe 10 and the controlling unit 16 could also be configured as one piece, with or without a display/screen 18, using either a wireless or USB connection to transfer data to a computer for post-processing and calculation purposes.

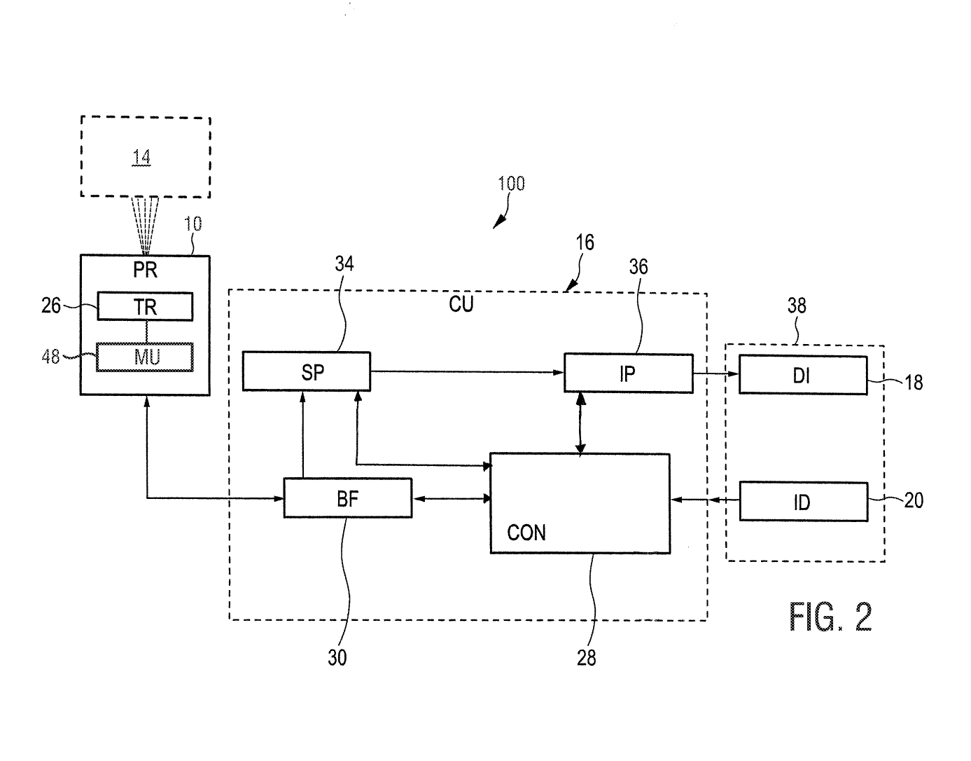

FIG. 2 shows a schematic block diagram of an ultrasound imaging system 100 according to an embodiment of the present invention. It shall be noted that this block diagram is used to illustrate the general concept and design of such an ultrasound system. In practice, the ultrasound imaging system 100 according to the present invention may slightly deviate from the design of this block diagram.

As already laid out above, the ultrasound imaging system 100 comprises an ultrasound probe (PR) 10, the controlling unit (CU) 16, the display (DI) 18 and the input device (ID) 20. The ultrasound probe 10 further comprises a single element ultrasound transducer (TR) 26 for transmitting and receiving ultrasound signals.

In general, the controlling unit (CU) 16 may comprise a central processing unit that may include analog and/or digital electronic circuits, a processor, microprocessor or the like to coordinate the whole image acquisition and provision. Further, the controlling unit 16 comprises a herein called image acquisition controller (CON) 28. However, it has to be understood that the image acquisition controller 28 does not need to be a separate entity or unit within the ultrasound imaging system 100. It can be a part of the controlling unit 16 and generally be hardware or software implemented. The current distinction is made for illustrative purposes only. The image acquisition controller 28 as part of the controlling unit 16 controls a beam former (BF) 30 and by this, what images of an examination area 14 are taken and how these images are taken. The beam former (BF) 30 generates voltages that drive the transducer element 26, determines parts repetition frequencies, it may scan, focus and apodize the transmitted beam and the reception or receive beam(s) and may further amplify, filter and digitize the echo voltage stream returned by the transducer element 26. Further, the image acquisition controller 28 may determine general scanning strategies. Such general strategies may include a desired volume acquisition rate, lateral extent of the volume, an elevation extent of the volume, maximum and minimum line densities, scanning line times and the line density itself. The beam former 30 further receives the ultrasound signals from the transducer element 26 and forwards them as image signals.

Further, the ultrasound system 100 comprises a signal processor (SP) 34 that receives said image signals. The signal processor 34 is generally provided for analog-to-digital-converting, digital filtering, for example, bandpass filtering, as well as the detection and compression, for example a dynamic range reduction, of the received ultrasound echoes or image signals. The signal processor 34 forwards image data.

Further, the ultrasound imaging system 100 comprises an image processor (IP) 36 that converts image data received from the signal processor 34 into display data finally shown on the display 18. In particular, the image processor 36 receives the image data, pre-processes the image data and may store it in an image memory (not explicitly shown). These image data are then further post-processed to provide images most convenient to the user via the display 18.

In the current case, in particular, the image processor 36 may form a two-dimensional image (B-mode) out of a multitude of one-dimensional A-scans acquired with the single element ultrasound transducer 26 during its movement within the probe housing. The image processor (IP) 36 is herein also denoted as image reconstruction unit 36.

A user interface is generally depicted with reference numeral 38 and comprises the display 18 and the input device 20. It may also comprise further input devices, for example, a mouse or further buttons which may even be provided on the ultrasound probe 10 itself.

FIG. 3 shows a preferred embodiment of the ultrasound probe 10. The ultrasound probe 10 comprises a probe housing 40 in which a single element ultrasound transducer 26 is arranged. The probe housing 40 usually comprises a handle 42 and a probe head 44. The probe head 44 of the probe housing 40 has a similar shape as a convex array transducer housing. At its front end it comprises a contact surface 46 for contacting a surface of the examination object (patient 12). Said contact surface 46 is a three-dimensional surface that preferably has an arc shape. From outside, the probe housing 40 may thus not be distinguished from a regular convex shaped multiple element array transducer as this known from the state of the art. The difference is however in the inside of the probe housing 40.

Instead of having a multiple element ultrasound transducer array, the ultrasound probe 10 according to the present invention preferably comprises only one single element ultrasound transducer 26. A transducer movement unit (MU) 48 (see FIG. 2) is arranged within the probe housing 40. This transducer movement unit (48) is configured to move the single element ultrasound transducer 26 relative to said probe housing 40 along a two-dimensional convex curved pathway during signal acquisition, as schematically illustrated with arrow 50 in FIG. 3B.

During signal acquisition, the single element transducer 26 is preferably automatically moved within the probe housing 40 in a very fast manner. A guiding rail (schematically illustrated by a dotted line 52) may be used for mechanically guiding the single element ultrasound transducer 26 along said convex curved pathway. That enables the single element transducer 26 to cover (during movement) a similar surface as a “regular” multi-element array probe.

During movement, the single element ultrasound transducer 26 may thus acquire several one-dimensional scan lines (A-mode) from which then a cone-shaped two-dimensional ultrasound image may be computed within the image reconstruction unit 36. A movement sensor 54 may be provided for sensing the movement and/or the position of the single element ultrasound transducer 26 relative to the probe housing 40. In this way the movement of the single element ultrasound transducer 26 is tracked exactly such that the position information of each scan line is known. The movement sensor 54 is preferably either arranged on the transducer element 26 or on the rail 52.

The single element transducer 26 may, for example, be used with a driving frequency of around 3.5 MHz.

The presented ultrasound probe 10 is preferably used for the detection and calculation/estimation of the amount of visceral adipose tissue (VAT) of a subject. Scanning will thus preferably be performed a few centimeters above the umbilicus, i.e. at the belly of the patient 12 (subject). Since in this measurement area relatively weak tissue (no bones) is usually present, pressure between the ultrasound probe 10 and the top surface to which it its applied is an important factor that should be controlled in order to achieve reproducible results. Otherwise, a too strong pressure with which the ultrasound probe 10 is applied to the subject could compress the tissue in the belly too much and could therefore falsify the fat measurements.

The ultrasound probe 10 therefore comprises at least one pressure sensor 56 that is arranged on the probe head 44 on or around the convex curved contact surface 46. A green and red blinking light 58, 58′ may be additionally provided to give the user a feedback if an adequate (correct) pressure is applied. It is to be noted that instead of blinking lights the feedback may also be produced in audible and/or tactile form. The blinking lights 58, 58′ are therefore generally denoted as feedback unit and could also be realized by a small loudspeaker or vibration sensor.

FIG. 4 shows three different embodiments of the ultrasound probe 10. It shall be noted that the features of these three different embodiments may also be combined without leaving the scope of the present invention. In all embodiments the convex curved contact surface 46 is symmetric with respect to an imaginary vertex line 60 that divides said contact surface 46 into two identical halves.

In the first embodiment illustrated in FIG. 4A the ultrasound probe 10 comprises two pressure sensors, a first pressure sensor 56 and a second pressure sensor 62. Both pressure sensors 56, 62 are preferably arranged on the imaginary vertex line 60 on or next to the contact surface 46. The first pressure sensor 56 is preferably arranged on the upper side of the contact surface 46 and the second pressure sensor 62 is preferably arranged on the lower side of the contact surface 46. The combination of these two pressure sensors 56, 62 does not only allow to check whether the applied pressure is within the desirable pressure range, but also allows to measure whether the probe head 44 is placed perpendicular in cranial/caudal direction. This may be seen by a simple comparison of the pressures measured with the first and the second pressure sensor 56, 62. If both pressures are equal, the probe head 44 is arranged exactly perpendicular. The larger the distance between these two pressure sensors 56, 62, the more sensitive and exact is the measurement.

The probe housing 40 may furthermore comprise two capacitive sensors 64, 64′ that are arranged on each lateral side of the contact surface 46. In order to transmit and receive the ultrasound signals correctly over the whole range of the contact surface 46, the contact surface 46 should have full contact with the top surface of the examination object. The two capacitive sensors 64, 64′ arranged on the lateral sides of the contact surface 46 allow to check whether the lateral sides of the probe head 44 are in contact with the examination object as well, i.e. whether the probe head 44 makes contact with the body of the subject 12 over the whole contact surface 46. It shall be noted that these capacitive sensors 64, 64′ may also be arranged in the corners of the contact surface 46. It is also possible to apply more than two capacitive sensors 64, 64′ at different positions of the contact surface 46. The contact surface 46 may also comprise an array of capacitive sensors that cover the whole contact surface 46.

In contrast to the first embodiment shown in FIG. 4A, the second embodiment illustrated in FIG. 4B comprises three instead of two pressure sensors. The first pressure sensor 56 remains at the same position (on the upper side of the contact surface 46). The second and the third pressure sensors 62, 66 are arranged on the lower side of the contact surface 46. While the first pressure sensor is arranged in the middle part of the contact surface 46 on the vertex 60, the second and the third pressure sensors 62, 66 are equally distanced from the vertex line 60. The provision of three pressure sensors 56, 62, 66 allows to check whether the probe head 44 is arranged perpendicular in both spatial directions. If the probe head 44 is arranged perpendicular in cranial/caudal direction, the pressure of the first pressure sensor 56 equals the sum of the pressures measured with the second and the third pressure sensors 62, 66. If the probe head 44 is perpendicular in lateral direction, the pressure sensed with the second pressure sensor 62 is equal with the pressure sensed with the third pressure sensor 66. To facilitate the handling for the user, the above-mentioned feedback unit 58 may again be used to give the user a feedback whether the probe head 44 is positioned correctly.

The third embodiment illustrated in FIG. 4C again comprises the three pressure sensors 56, 62, 66 and the capacitive sensors 64, 64′. It additionally comprises a displacement sensor 68 that senses whether the probe housing 40 is moved with respect to the examination object 12 or not. This displacement sensor 68 is preferably realized as an optical sensor. It is preferably arranged on the imaginary vertex line 60. However, it may also be arranged at another position of the probe head 44.

As it has been already mentioned above, the primary use of the ultrasound imaging system 100 is to quantify/estimate the VAT. It shall, however, be noted that the presented ultrasound imaging system is not restricted to this use and may also be used for other purposes.

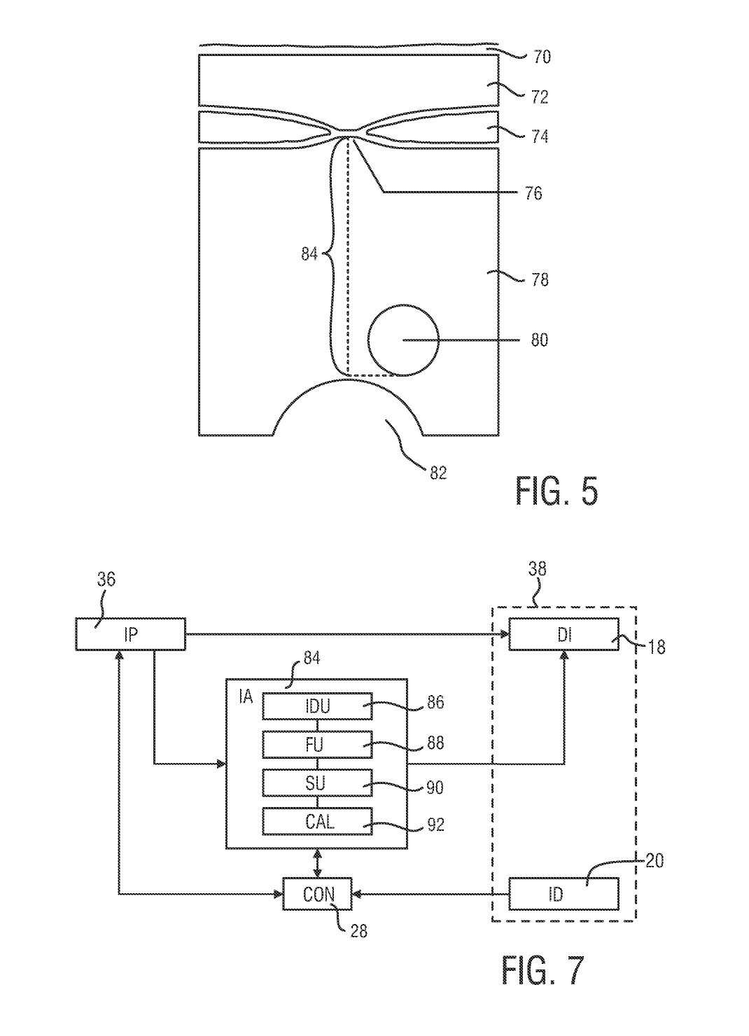

The estimation/quantification of the VAT is primarily based on the intra-abdominal diameter (IAD). FIG. 5 shows a schematic cross section of the human abdominal region. The top layer represents the skin 70. Further below is the subcutaneous adipose tissue (SAT) 72, the rectus abdominis muscle 74 including the Linea Alba 76. The human VAT surrounds the internal organs and is herein schematically denoted with reference numeral 78. Reference numerals 80 and 82 denote the Aorta and the vertebral body. The above-mentioned IAD is denoted in FIG. 5 by reference numeral 84 and describes the distance between the Linea Alba 76 and the posterior wall of the Aorta 80.

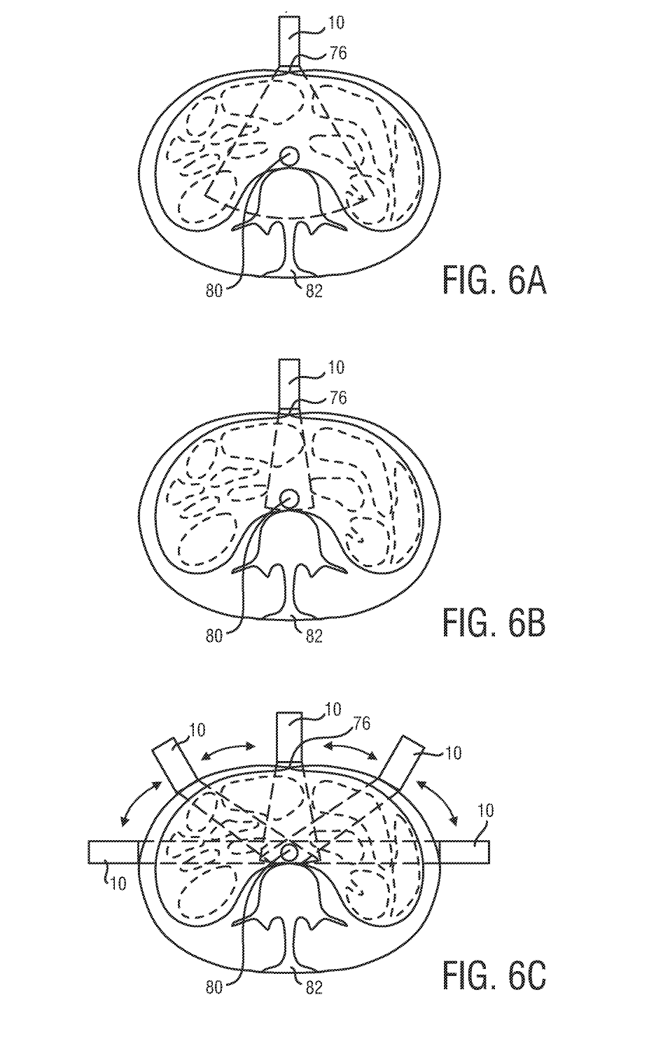

The handling of the presented device and the measurement of the VAT will be explained in the following with reference to FIGS. 6 to 8.

FIG. 6 schematically illustrates a preferred usage and scanning procedure. In a first step, the ultrasound probe 10 is placed just above the umbilicus at the L3-L4 level of the spine. The above-mentioned pressure sensors 56, 62, 66 may help to apply the “correct” pressure and to orientate the probe head 44 “correctly”, i.e. as perpendicular as possible. Signal acquisition may then be started, for example, by pressing a button. This will cause the single transducer element 26 to move along the two dimensional convex curved array as explained above with reference to FIG. 3. During movement of the single transducer element 26 along the convex pathway, the epigastric/umbilical region at the L3-L4 level of the spine is imaged (see FIG. 6A). In the next phase the Aorta 80 is identified within the resulting ultrasound image using image analysis (explained below in detail). As soon as the Aorta 80 is identified within the ultrasound image, the single element ultrasound transducer 26 will be focused on the Aorta 80 (FIG. 6B). The user can now slide the ultrasound probe 10 over the belly in the horizontal plane (see FIG. 6C). During this movement several scans are taken, while the Aorta 80 is still focused and taken as a reference point. In this way the complete epigastric/umbilical region at the L3-L4 level of the spine is imaged, such that a two-dimensional image of the complete region may be reconstructed. A further image analysis then allows to derive the position of the Linea Alba 76 and the posterior wall of the Aorta 80, such that the IAD 84 may be determined and the amount of VAT may be calculated/estimated.

Instead of scanning the whole epigastric region, the ultrasound probe 10 may also be kept still at a single position, e.g. at the position shown in FIG. 6A, such that only a part of the epigastric region including the Linea Alba 76 and the Aorta 80 is imaged. If several image sequences (a video) are taken over time at this position of the ultrasound probe 10, the IAD may be derived therefrom as well. This could, for example, be done by averaging the distance of the IAD between several respiration cycles and Aorta pulsations. The above-mentioned displacement sensor 68 may thereby help to account for eventual displacement errors.

FIG. 7 illustrates a schematic block diagram of an embodiment of the present invention. It is to be noted that this block diagram illustrates the right part of the block diagram shown in FIG. 2. In contrast to the block diagram shown in FIG. 2, an additional imaging analysis unit 84 is provided. This imaging analysis unit 84 may either be hardware or software based. It may also be comprised in one of the other components that were explained above with reference to FIG. 2. The imaging analysis unit 84 preferably receives the ultrasound images after they have been post-processed within the image processor 36. The image analysis unit 84 preferably comprises an identification unit (IDU) 86, a focusing unit (FU) 88, a segmentation unit (SU) 90 and a calculation unit (CAL) 92.

The identification unit 86 is configured to identify a reference point, in particular the Aorta 80, within the reconstructed ultrasound image(s). The focusing unit 88 is configured to focus the single element ultrasound transducer 26 on said reference point during the movement of the ultrasound probe 10 relative to the examination object 12. The segmentation unit 90 is configured to segment the reconstructed ultrasound images and to identify a position of the Linea Alba 76 and the Aorta 80 within the ultrasound image in order to derive the intra-abdominal diameter (IAD). The calculation unit 92 is configured to calculate the amount of VAT based on the derived IAD. The calculated amount of VAT may finally be displayed on the display 18.

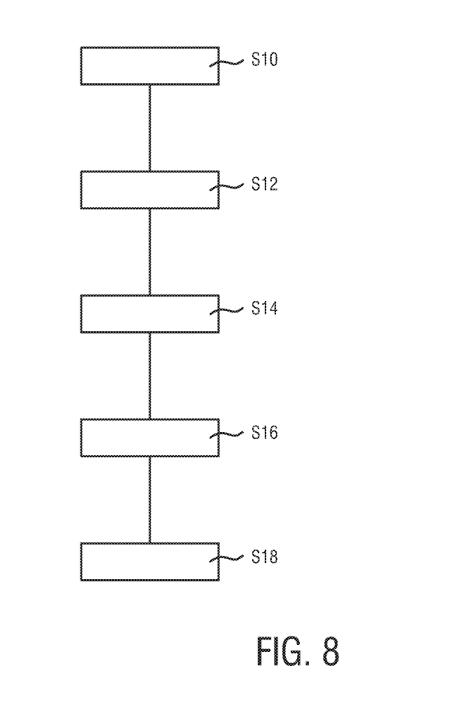

FIG. 8 illustrates the method again in a schematic block diagram. In the first step (S10) ultrasound signals are received from the above-mentioned single-element ultrasound transducer 26 that is during signal acquisition automatically moved within the probe housing 40 of the ultrasound probe 10 along the convex curved pathway. In the following step S12 the ultrasound image is reconstructed from the ultrasound signals received from the ultrasound transducer 26. In step S14 the reconstructed ultrasound image of the abdominal ultrasound scan of the subject 12 is segmented. The image analysis unit may apply an image analysis algorithm that is adopted to derive the location of the Linea Alba and the Aorta (step S16). In a first step this usually comprises a region of interest (ROI) selection. An ROI for the Linea Alba detection and an ROI for the Aorta detection can be first selected in the original input ultrasound image. The ROIs can be selected based on prior knowledge of the anatomical structure and ultrasound imaging. For example, the Linea Alba 76 will lie in the upper part of the ultrasound image and the Aorta 80 is represented by the largest pulsating object in the middle part of the image.

To increase the contrast of the gained ultrasound image, image enhancement techniques are applied to the selected ROIs in a next step. For example, a histogram equalization can be adopted to enhance the contrast by spreading out the most frequent pixel intensity values. Such an image enhancement technique is, for example, known from S. H. Contreras Ortiz, et. al.: “Ultrasound image enhancement: A review”, Biomedical Signal Processing and Control, 7(5): 419-428, 2012.

Object localization techniques are then adopted to localize the Linea Alba and the Aorta in the processed ROIs (step S16). Different methods exist for object localization in computer vision and image analysis areas. In one embodiment, machine learning based methods can be used. Given many positive samples (e.g., the image patch of Linea Alba) and negative samples (e.g., the image patch not corresponding to Linea Alba), machine learning techniques are used to train a detector for Linea Alba or Aorta. Such a machine learning technique is exemplarily described in P. Viola and M. Jones: “Rapid Object Detection using a Boosted Cascade of Simple Features”, CVPR conference 2011. With the trained detector, a given ROI is scanned at multiple scales and multiple positions, to check whether the Linea Alba (or Aorta) exists in the ROI and if yes, to find the location.

In another embodiment, a deformable template model can be considered (see e.g. A. K. Jain, et al.: “Object Matching Using Deformable Templates”, IEEE Trans. Pattern Analysis and Machine Intelligence, 18(3): 267-278, 1996). A prototype template is defined for the Linear Alba or the Aorta based on the prior knowledge. Given a ROI, the template is applied to multiple positions (at multiple scales), to see whether the Linea Alba (or Aorta) can be matched. Based on template matching, the Linea Alba or Aorta may be localized.

In case multiple ultrasound images (or video) are acquired, information from multiple frames can be combined to improve the accuracy and robustness. In one embodiment, the detection results in multiple images are combined (e.g., averaging) to derive the final location of the Linea Alba or Aorta, which is a decision-level fusion. In another embodiment, feature-level fusion can be used, that is, image content (or features) of multiple images are considered in the object localization.

If the Linea Alba 76 and the Aorta 80 are finally detected within the ultrasound image, the IAD 84 can be derived (step S16). The amount of visceral body fat may then be calculated based on the derived IAD in the last step S18. Several calculation methods may be used thereto.

Armellini, F et al: “Measured and predicted total and visceral adipose tissue in women. Correlations with metabolic parameters”, International Journal of Obesity (1994) 18, 641-647 concluded the usage of the IAD to be the best method to predict VAT, but equations for single subject predictions provided not to give sufficient accuracy. For this reason Armellini, F et al give an equation which also takes waist circumference into account:

VAT=−117+1.73 US+1.43 waist+1.51 age;

wherein US is ultrasound measurement of distance between abdominal muscle and aorta.

The main issue of this calculation is the lack of standardization for pressure, breathing and aorta pulsations (all factors which are standardized for according to the present invention, either using hardware or software algorithm solutions). A preferred calculation according to the present invention is therefore:

VAT=\alpha+\beta*IAD

wherein alpha includes several factors, including gender, age, etc. and beta is a scaling factor found from experiments. Instead of this linear equation also more complex equations could be used.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. An ultrasound probe for an ultrasound imaging system, comprising:
a probe housing having a three-dimensional convex curved contact surface for contacting a surface of an examination object, wherein the contact surface is symmetric with respect to an imaginary vertex line that divides the contact surface in two identical halves,
a single element ultrasound transducer for transmitting and receiving ultrasound signals,
a transducer movement unit arranged within the probe housing for moving the single element ultrasound transducer relative to said probe housing along a two-dimensional convex curved pathway during signal acquisition.

2. The ultrasound probe according to claim 1, wherein said transducer movement unit comprises a convex shaped guiding rail for mechanically guiding the single element ultrasound transducer relative to the probe housing along the convex curved pathway.

3. The ultrasound probe according to claim 1, further comprising a displacement sensor for sensing a movement and/or position of the single element ultrasound transducer and/or the probe housing.

4. The ultrasound probe according to claim 1, further comprising at least one pressure sensor for sensing a pressure with which the probe housing is pressed against a surface of an examination object.

5. (canceled)

6. The ultrasound probe according to claim 1, further comprising two pressure sensors for sensing a pressure with which the probe housing is pressed against a surface of an examination object, said two pressure sensors being arranged on the imaginary vertex line and spaced apart from each other.

7. The ultrasound probe according to claim 6, wherein both pressure sensors are arranged on or within the contact surface on two opposing sides of the contact surface, and wherein a distance between the two pressure sensors substantially equals a width of the contact surface.

8. The ultrasound probe according to claim 1, further comprising three pressure sensors that are arranged on or within the contact surface for sensing a pressure with which the probe housing is pressed against a surface of an examination object, wherein a first pressure sensor is arranged on the imaginary vertex line, and wherein a second and a third pressure sensor are spaced apart from the first pressure sensor and equally spaced apart from said vertex line.

9. The ultrasound probe according to claim 4, further comprising a visual, audible and/or tactile feedback unit for providing a feedback to a user about the pressure(s) measured with said pressure sensor(s).

10. The ultrasound probe according to claim 1, further comprising two capacitive sensors arranged on two opposing lateral sides of the contact surface for sensing if the probe housing makes contact with an examination object over the whole contact surface, wherein a distance between the two capacitive sensors substantially equals a length of said contact surface.

11. An ultrasound imaging system comprising:
an ultrasound probe according to claim 1, and
an image reconstruction unit for reconstructing an ultrasound image from the received ultrasound signals.

12. The ultrasound imaging system according to claim 11, further comprising:
an identification unit for identifying a reference point within the reconstructed ultrasound image, and
a focussing unit for focussing the single element ultrasound transducer on the reference point during a movement of the ultrasound probe relative to an examination object.

13. The ultrasound imaging system according to claim 11, further comprising:
an segmentation unit for segmenting an ultrasound image of an abdominal ultrasound scan of a subject and for identifying a position of a Linea Alba and an Aorta within said ultrasound image in order to derive an intra-abdominal diameter (IAD), and
a calculation unit for calculating an amount of visceral body fat based on the derived IAD.

14. A method of determining an amount of visceral body fat of a subject from an abdominal ultrasound scan, wherein the method comprises the steps of:
receiving ultrasound signals from a single element ultrasound transducer that is during signal acquisition automatically moved within a probe housing of an ultrasound probe along a convex curved pathway, said probe housing having a three-dimensional convex curved contact surface or contacting a surface of an examination object, wherein the contact surface symmetric with respect to an imaginary vertex line that divides the contact surface in two identical halves,
reconstructing an ultrasound image from the received ultrasound signals,
segmenting the ultrasound image of the abdominal ultrasound scan of the subject (12),
identifying a position of a Linea Alba and an Aorta within said ultrasound image in order to derive an intra-abdominal diameter (IAD), and
calculating the amount of visceral body fat based on the derived IAD.

15. Computer program comprising program code means for causing a computer to carry out the steps of the method as claimed in claim 14 when said computer program is carried out on a computer.
